(19) United States
(12) Patent Application Publication
(10) Pub. No.: US 2025/0149119 A1
(43) Pub. Date: May 8, 2025

(54) METHOD OF CONSTRUCTING A SPATIALLY BARCODED SURFACE

(71) Applicant: Cellanome, Inc., Foster City, CA (US)

(72) Inventor: Tarun Kumar KHURANA, Fremont, CA (US)

(21) Appl. No.: 18/952,420

(22) Filed: Nov. 19, 2024

Related U.S. Application Data
(63) Continuation of application No. PCT/US2023/023004, filed on May 19, 2023.
(60) Provisional application No. 63/344,380, filed on May 20, 2022.

(51) Int. Cl. G16B 30/20

(52) U.S. Cl. CPC G16B 30/20

(57) ABSTRACT

The systems, devices, and methods described herein are directed to methods for producing a spatially barcoded surface using combinatorial synthesis techniques.

### CROSS-REFERENCE TO RELATED APPLICATIONS

This application is a Continuation Application of International Application No. PCT/US2023/023004, filed May 19, 2023, which claims the benefit of U.S. Provisional Application No. 63/344,380, filed May 20, 2022, which is incorporated herein by reference in its entirety.

### BACKGROUND

Molecular tagging has a long history in analytical biochemistry and molecular biology, e.g. Church U.S. Pat. No. 4,942,124; Spitzer et al, Cell, 165(4): 780-791 (2016); Giese, Trends in Analytical Chemistry, 2(7): 166-168 (1983); Hardenbol et al, Nature Biotechnology, 21: 673-678 (2003); Brenner et al, U.S. Pat. No. 7,537,897; Fan et al, Science, 347 (6222): 1258367-1 (2015); Macevicz, U.S. patent publication US2005/0250147; Morris et al, European patent publication 0799897A1; Wallace, U.S. Pat. No. 5,981,179; and the like. Recently, such techniques have been expanded to include the use of spatially distributed oligonucleotide barcodes for identifying and studying spatial variations in biological processes, such as tissue-wide gene expression, e.g. Stahl et al, Science, 353(6294): 78-82 (2016); Salmen et al, Nature Protocols, 13: 2501-2534 (2018); Frisen et al, U.S. Pat. No. 9,593,365; and the like. However, cost effective synthesis of spatial barcodes with known sequences, control of spatial barcode distributions, and densities for resolving cellular and subcellular processes have been a challenge. This challenge has been addressed with only partial success by a plethora of different approaches, e.g. Horgan et al, International patent publication, WO/2022/013094; Liu et al, Cell, 183: 1665-1681 (2020); Cho et al, bioRxiv (https://doi.org/10.1101/2021.01.25.427004); Chen et al (https://doi.org/10.1101/2021.01.17.427807); Delly et al, Scientific Reports, 11: 10857 (2021); Rodriques et al, Science, 363(6434): 1463-1467 (2019); and the like. The field of spatial barcode construction would be advanced by the availability of a cost effective spatial barcoding method.

### SUMMARY

The systems, devices, and methods described herein are directed to making spatially barcoded surfaces and nucleic acid molecules using combinatorial techniques. In some embodiments, methods of making a spatially barcoded surface comprises: (a) providing a solid support comprising a surface; (b) synthesizing a plurality of arrays of first oligonucleotides, each array comprising a plurality of discrete reaction sites and each first oligonucleotide attached to the surface by its 5′-end, wherein (i) each first oligonucleotide has a barcode segment comprising a different barcode sequence from those of other first oligonucleotides (ii) first oligonucleotides having different barcode sequences are attached at different reaction sites, (iii) the plurality of arrays are arranged in orthogonal rows and columns; and (iv) each first oligonucleotide occupies a reaction site at a known surface location; (c) partitioning the surface into rows by sealingly attaching to the surface a first channel template comprising a plurality of channels; (d) reacting second oligonucleotides loaded into each channel of the first channel template with the surface or prior conjugate surface oligonucleotides to form conjugate surface oligonucleotides or spatial barcode oligonucleotides, wherein each different channel comprises a second oligonucleotide comprising a second barcode segment comprising a different barcode sequence from those of second oligonucleotides of different channels; wherein (A) the rows of the plurality of arrays are at least partially coincident with the rows of the first channel template, and (B) each spatial barcode oligonucleotide uniquely identifies the spatial location of its reaction site on the surface. In some embodiments, in step (d) second oligonucleotides are reacted directly with the surface, while in other embodiments, in step (d), second oligonucleotides are reacted with oligonucleotides already attached to the surface. In the latter embodiments, the oligonucleotides already attached to the surface are referred to as “conjugate surface oligonucleotides,” which may be concatenations of first, second or third oligonucleotides from previous steps. That is, conjugate surface oligonucleotides are partially completed oligonucleotide precursors to spatial barcode oligonucleotides. In some embodiments, a step (d) may be the final attachment of a barcode segment (which may be a first, second or third oligonucleotide, depending on the embodiment) resulting in the final desired “spatial barcode oligonucleotide.”

In some embodiments, methods of making a spatially barcoded surface comprises: (a) providing a surface; (b) synthesizing a plurality of arrays of first oligonucleotides, each array comprising a plurality of discrete reaction sites and each first oligonucleotide attached to the surface by its 5′-end, wherein (i) each first oligonucleotide has a barcode segment comprising a different barcode sequence from those of other first oligonucleotides (ii) first oligonucleotides having different barcode sequences are attached at different reaction sites, (iii) the plurality of arrays are arranged in orthogonal rows and columns; and (iv) each first oligonucleotide occupies a reaction site at a known surface location; (c) partitioning the surface into rows by sealingly attaching to the surface a first channel template comprising a plurality of channels; (d) reacting second oligonucleotides loaded into each channel of the first channel template with the surface or prior conjugate surface oligonucleotides to form conjugate surface oligonucleotides or spatial barcode oligonucleotides, wherein each different channel comprises a second oligonucleotide comprising a second barcode segment comprising a different barcode sequence from those of second oligonucleotides of different channels; (e) partitioning the surface into columns by sealingly attaching to the surface a second channel template comprising a plurality of channels; (f) reacting third oligonucleotides loaded into each channel of the second channel template with the surface or prior conjugate surface oligonucleotides to form conjugate surface oligonucleotides or spatial barcode oligonucleotides, wherein each different channel of the second channel template comprises a third oligonucleotide comprising a third barcode segment comprising a different barcode sequence from those of third oligonucleotides of different channels of the second channel template; and wherein (A) the rows of the plurality of arrays are at least partially coincident with the rows of the first channel template and the columns of the plurality of arrays are at least partially coincident with the columns of the second channel template, and (B) each spatial barcode oligonucleotide uniquely identifies the spatial location of its reaction site on the surface. As noted above, the term “conjugate surface oligonucleotide” refers to a partially completed oligonucleotide precursor to a spatial barcode oligonucleotide.

In some embodiments, methods for making spatially barcoded nucleic acid molecules comprise (a) providing a solid support comprising a surface; (b) capturing nucleic acid molecules on the surface and transcribing the captured nucleic acid molecules into complementary DNAs (cDNAs) attached to the surface; (c) synthesizing a plurality of arrays of first oligonucleotides at different reaction sites on the surface, wherein (i) the plurality of arrays are arranged in orthogonal rows and columns; (ii) each array comprises a plurality reaction sites each comprising a first oligonucleotide comprising a first barcode segment with a different barcode sequence whenever located in a different reaction site; and (iii) each first oligonucleotide occupies a reaction site at a known surface location; (d) reacting second oligonucleotides with the surface or oligonucleotides or cDNAs thereon by sealingly attaching to the surface a first channel template comprising a plurality of channels, wherein each different channel comprises a second oligonucleotide comprising a second barcode segment comprising a different barcode sequence from those of second oligonucleotides of different channels; and (e) reacting third oligonucleotides with the surface or oligonucleotides or cDNAs thereon by sealingly attaching to the surface a second channel template comprising a plurality of channels, wherein each different channel of the second channel template comprises a third oligonucleotide comprising a third barcode segment comprising a different barcode sequence from those of third oligonucleotides of different channels; wherein the channels of the first and second channel templates align with the orthogonal rows and columns of the arrays and wherein the first, second and third oligonucleotides attached to a cDNA form a spatial barcode that identifies a spatial location of the cDNA on the surface.

In an aspect, provided herein is a method of generating a spatially barcoded surface, comprising: (a) providing a solid support comprising a surface, wherein the surface comprises a plurality of arrays arranged on the surface, wherein each array comprises a plurality of reaction sites, wherein each reaction site comprises a reaction site oligonucleotide with a barcode sequence unique to the reaction site in which it is located; (b) partitioning the surface into one or more channels by coupling to the surface a channel template comprising a plurality of channels; and (c) loading a plurality of channel oligonucleotides into the plurality of channels such that at least one channel oligonucleotide couples to the reaction site oligonucleotide in each array, wherein each channel oligonucleotide comprises a barcode sequence unique to the channel in which it is located.

In some cases, the method further comprises: (a) partitioning the surface into one or more orthogonal channels by coupling to the surface an additional channel template comprising a plurality of orthogonal channels, wherein the one or more orthogonal channels are orthogonal to the one or more channels; and (c) loading a plurality of orthogonal channel oligonucleotides into the plurality orthogonal channels such that at least one orthogonal channel oligonucleotide couples to the channel oligonucleotide in each array, wherein each orthogonal channel oligonucleotide comprises a barcode sequence unique to the orthogonal channel in which it is located.

In some cases, wherein the plurality of arrays are arranged in rows and columns. In some cases, the one or more channels at least partially coincide with the rows, and wherein the one or more orthogonal channels at least partially coincide with the columns. In some cases, the one or more channels at least partially coincide with the columns, and wherein the one or more orthogonal channels at least partially coincide with the rows.

In some cases, the coupling in (b) comprises sealingly attaching to the surface the channel template comprising the plurality of channels. In some cases, the coupling in (a) comprises sealingly attaching to the surface the additional channel template comprising the plurality of orthogonal channels.

In some cases, each of the arrays of the plurality are the same. In some cases, an array of the plurality of arrays comprises a pitch between the reaction sites in the range of from 50-500 m, and wherein the reaction sites each have a diameter in the range of from 30-300 m. In some cases, an array of the plurality of arrays comprises a density of reaction sites in the range of from 50 to 200 reaction sites per mm².

In some cases, the coupling of the at least one channel oligonucleotide to the reaction site oligonucleotide in each array in (c) comprises extending the at least one channel oligonucleotide using a DNA polymerase. In some cases, the coupling of the at least one channel oligonucleotide to the reaction site oligonucleotide in each array in (c) comprises ligating the at least one channel oligonucleotide to the reaction site oligonucleotide in each array.

In some cases, surface further comprises a capture probe attached thereto, and wherein the method further comprises capturing a sample nucleic acid with the capture probe and extending the capture probe using the captured sample nucleic acid as a template. In some cases, any of the reaction site oligonucleotides, channel oligonucleotides, or orthogonal channel oligonucleotides comprise the capture probe.

In another aspect, provided herein is a method of making a spatially barcoded surface, comprising: (a) providing a solid support comprising a surface, wherein the surface comprises a plurality of arrays, wherein an array of the plurality of arrays comprises at least two reaction sites, wherein a first reaction site of the at least two reaction sites comprises a first reaction site oligonucleotide comprising a first reaction site barcode sequence, and wherein a second reaction site of the at least two reaction sites comprises a second reaction site oligonucleotide comprising a second reaction site barcode sequence, and wherein the first barcode sequence is different from the second barcode sequence; (b) coupling to the surface a channel template comprising a first channel and a second channel; (c) loading a first channel oligonucleotide into the first channel; and (d) loading a second channel oligonucleotide into the second channel, wherein, subsequent to the loading of (c), the first channel oligonucleotide couples to the first reaction site oligonucleotide, and wherein, subsequent to the loading of (d), the second channel oligonucleotide couples to the second reaction site oligonucleotide, and wherein the first channel oligonucleotide comprises a third barcode sequence and the second channel oligonucleotide comprises a fourth barcode sequence, and wherein the third barcode sequence is different from the fourth third barcode sequence.

In some cases, the method further comprises: (a) coupling to the surface an orthogonal channel template comprising a first orthogonal channel and a second orthogonal channel; (b) loading a first orthogonal channel oligonucleotide into the first channel; and (c) loading a second orthogonal channel oligonucleotide into the second channel, wherein, subsequent to the loading of (b), the first orthogonal channel oligonucleotide couples to the first channel oligonucleotide, and wherein, subsequent to the loading of (c), the second orthogonal channel oligonucleotide couples to the second channel oligonucleotide, and wherein the first orthogonal channel oligonucleotide comprises a fifth barcode sequence and the second orthogonal channel oligonucleotide comprises a sixth barcode sequence, and wherein the fifth barcode sequence is different from the sixth third barcode sequence.

In some cases, the plurality of arrays are arranged in rows and columns. In some cases, the first channel and the second channel at least partially coincide with the rows, and wherein the first orthogonal channel and the second orthogonal channel at least partially coincide with the columns. In some cases, the first channel and the second channel at least partially coincide with the columns, and wherein the first orthogonal channel and the second orthogonal channel at least partially coincide with the rows.

In some cases, the coupling in (b) comprises sealingly attaching to the surface the channel template comprising the first channel and the second channel. In some cases, the coupling in (a) comprises sealingly attaching to the surface the orthogonal channel template comprising the first orthogonal channel and the second orthogonal channel.

In some cases, each of the arrays of the plurality are the same. In some cases, an array of the plurality of arrays comprises a pitch between the reaction sites in the range of from 50-500 m, and wherein the reaction sites each have a diameter in the range of from 30-300 m. In some cases, an array of the plurality of arrays comprises a density of reaction sites in the range of from 50 to 200 reaction sites per mm².

In some cases, the coupling of the first channel oligonucleotide to the first reaction site oligonucleotide comprises extending the first channel oligonucleotide using a DNA polymerase. In some cases, the coupling of the first channel oligonucleotide to the first reaction site oligonucleotide comprises ligating the first channel oligonucleotide to the first reaction site oligonucleotide. In some cases, the coupling of the first orthogonal channel oligonucleotide to the first channel oligonucleotide comprises extending the first channel oligonucleotide using a DNA polymerase. In some cases, the coupling of the first orthogonal channel oligonucleotide to the first channel oligonucleotide comprises ligating the first orthogonal channel oligonucleotide to the first channel oligonucleotide.

In some cases, the surface further comprises a capture probe attached thereto, and wherein the method further comprises capturing a sample nucleic acid with the capture probe and extending the capture probe using the captured sample nucleic acid as a template. In some cases, any of the first or second reaction site oligonucleotides, the first or second channel oligonucleotides, or the first or second orthogonal channel oligonucleotides comprise the capture probe.

In another aspect, provided herein is a flow cell, comprising: one or more arrays, wherein an array of the one or more arrays is located at an intersection of a row and a column on a surface of the flow cell, wherein the array comprises one or more reaction sites, and wherein a reaction site of the one or more reaction sites comprises: (a) a first oligonucleotide sequence unique to a spatial location of the reaction site within the array, (b) a second oligonucleotide unique to the row, and (c) a third oligonucleotide unique to the column. 59. In some embodiments, the array, the row, the column, or any combination thereof is configured to receive the first oligonucleotide, the second oligonucleotide, the third oligonucleotide, or any combination thereof. In some embodiments, the array comprises a pitch between the reaction site and a second reaction site in the range of from 50-500 μm, and wherein the reaction site comprises a diameter in the range of from 30-300 μm. In some embodiments, the one or more reaction sites comprise a density in the range of from 50 to 200 reaction sites per mm². In some embodiments, the one or more reaction sites comprise one or more capture probes. In some embodiments, the flow cell further comprises a second array, wherein the second array is located at a second intersection of a second row and a second column, wherein the second array comprises one or more second reaction sites, and wherein a second reaction site of the one or more second reaction sites comprises: a fourth oligonucleotide sequence unique to a spatial location of the second reaction site within the second array; a fifth oligonucleotide unique to the second row; and a six oligonucleotide unique to the second column. In some embodiments, the spatial location of the second reaction site within the second array corresponds to the spatial location of the reaction site within the array of claim 58, and wherein the first oligonucleotide sequence and the fourth oligonucleotide sequence are the same.

### BRIEF DESCRIPTIONS OF THE DRAWINGS

FIG. 1A illustrates one format of a combinatorial spatial barcode which may be used with the systems and methods described herein.

FIG. 1B illustrates an embodiment of the systems and methods described herein in which a final barcode segment is attached by tagmentation.

FIGS. 2A and 2B illustrate an embodiment for producing a spatially barcoded surface.

FIGS. 2C-2E illustrate embodiments for producing spatially barcoded surfaces in which channels are partially coincident with arrays of first oligonucleotide either by adjusting the spot pattern of the arrays (FIG. 2D), employing multiple channel templates that off-set channel positions (FIG. 2E).

FIG. 2F illustrates the production of combinatorial barcodes in one dimension by employing a plurality of channel templates with different channel widths and off-sets.

FIG. 3 illustrates an appliance for creating channels for applying reagents to rows or columns of spotted arrays on a surface.

### DETAILED DESCRIPTION

The practice of the systems and methods described herein may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, monoclonal antibodies, antibody display systems, cell and tissue culture techniques, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Renault and Duchateau, Editors, Site-directed Insertion of Transgenes (Springer, Heidelberg, 2013); Lutz and Bornscheuer, Editors, Protein Engineering Handbook (Wiley-VCH, 2009); and the like. Guidance for selecting materials and components to carry out particular functions may be found in available treatises and references on scientific instrumentation including, but not limited to, Moore et al, Building Scientific Apparatus, Third Edition (Perseus Books, Cambridge, MA); Hermanson, Bioconjugate Techniques, 3rd Edition (Academic Press, 2013); and like references.

The systems and methods described herein are directed to making or generating spatially barcoded surfaces and their use to analyze molecules, especially nucleic acid molecules, of biological cells disposed on such surfaces. The systems and methods described herein are also directed to spatially barcoding nucleic acid molecules disposed or captured on a surface. Spatial barcodes may be combinatorial in the sense that each barcode is a combination of at least three segments: two segments that identify the position of an array on the surface and a third segment that identifies the position of the barcode oligonucleotide, or the nucleic acid molecule it is attached to, within the array. Moreover, in some embodiments, the final library of spatial barcodes comprises every combination of the possible sequences of the first, second and third barcode segments. Thus, in embodiments employing three barcode segments, the number of first oligonucleotides (each containing a first barcode segment) in an array, the number of channels for delivering second oligonucleotides (each containing a second barcode segment) and the number of channels for delivering third oligonucleotides (each containing a third barcode segment) determines the total number of different barcodes on a surface. For example, for an array of 384 first oligonucleotides, a first channel template of 50 channels and a second channel template of 50 channels, a surface may have 980,000 (=50×50×384) different barcodes. Channel templates and gaskets to sealingly attach templates to a surface may be made using fabrication techniques employed for microfluidics devices.

A wide variety of surfaces may be used with the systems and methods described herein. In some embodiments, surfaces are two-dimensional planar surfaces of a solid support material. Such solid support materials may comprise non-porous solids that may be derivatized with conventional functionalities by which oligonucleotides may be attached (e.g. Devor et al, Integrated DNA Technologies (2005), or the like). In some embodiments, such solid support materials may comprise glass, plastic, silicon, metal oxides, or the like. In some embodiments, a surface is a glass support material, such as a glass slide.

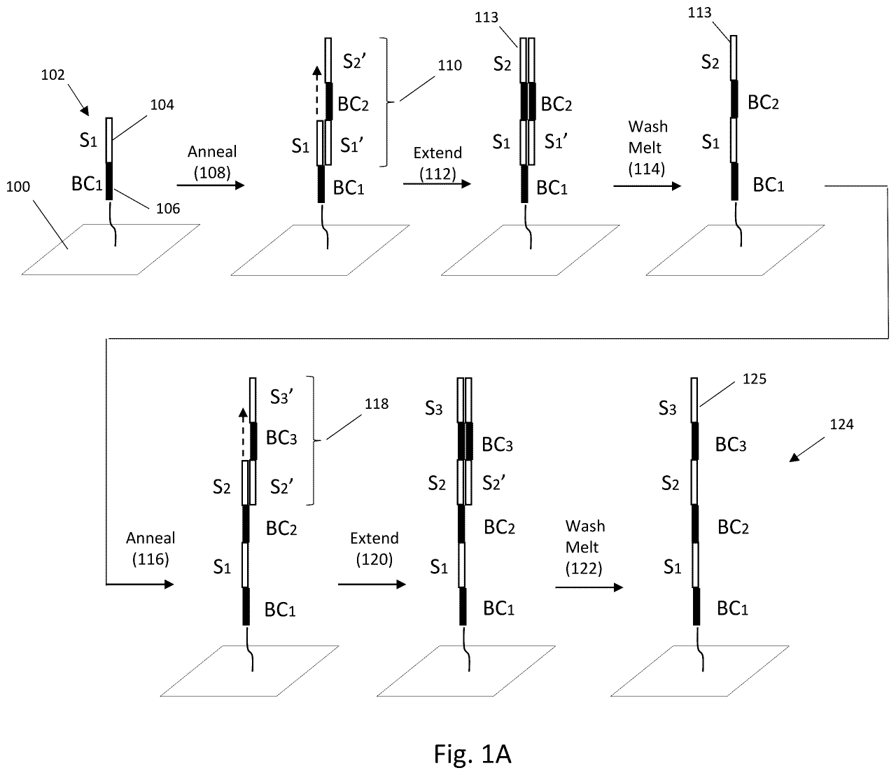

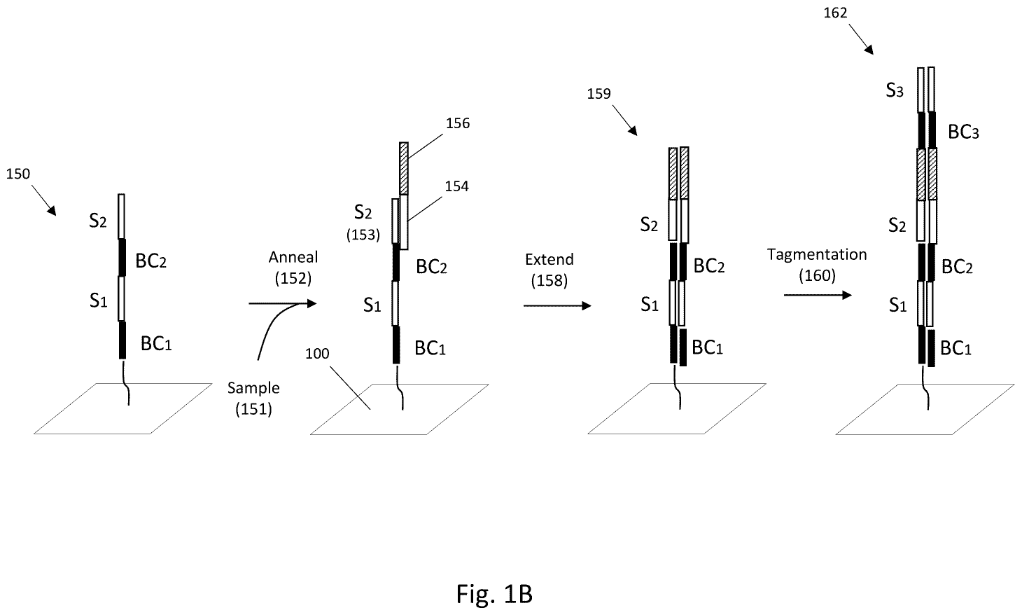

In various embodiments of the systems and methods described herein, barcode segments may be attached before and/or after capture and replication of nucleic acid molecules from samples. In other words, the order in which barcode segments and sample nucleic acids are assembled on a surface may vary so that the ordering of cDNA (transcribed from a captured nucleic acid) and the barcode segments making up a spatial barcode may be selected. In different embodiments, such ordering (from the surface) may be as follows: -cDNA-BC₁-BC₂-BC₃; BC₁-cDNA-BC₂-BC₃; BC₁-BC₂-cDNA-BC₃; or BC₁-BC₂-BC₃-cDNA, where BC₁, BC₂ and BC₃ represent the first, second and third oligonucleotides (containing the first, second and third barcode segments), respectively. The assembly of first, second and third oligonucleotides to produce a barcoded surface or the assembly of cDNAs, and first, second and third oligonucleotides to produce a surface with spatially barcoded cDNAs is accomplished using conventional methods for linking nucleic acid molecules to one another or to surfaces, which are exemplified for the embodiments described in FIGS. 1A and 1B.

Although embodiments are disclosed showing the formation of spatial barcodes comprising two or three barcode segments, the systems and methods described herein may also include combinatorial spatial barcodes of a plurality of barcode segments. In some embodiments, combinatorial spatial barcodes comprise from 3 to 6 barcode segments; or from 3 to 5 segments; or from 3 to 4 segments. In some embodiments, combinatorial spatial barcodes having greater than three barcode segments may be produced by applying additional steps of partitioning and reacting using (or reusing) channel templates loaded with oligonucleotides comprising different combinations of barcode sequences.

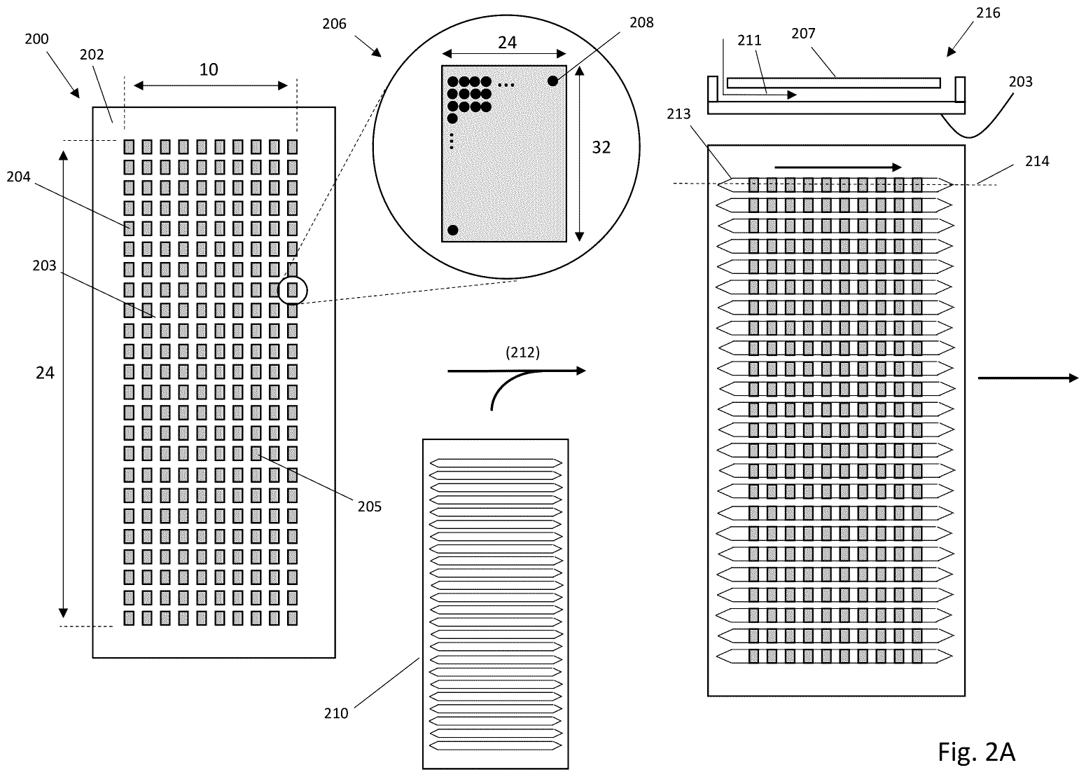

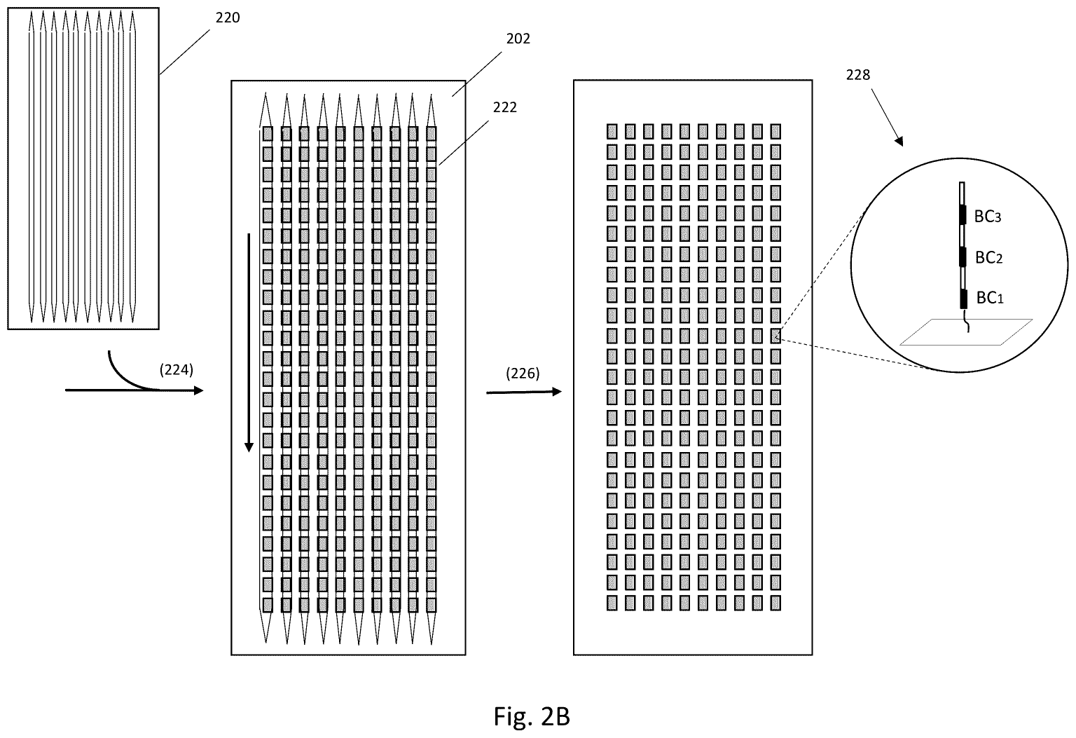

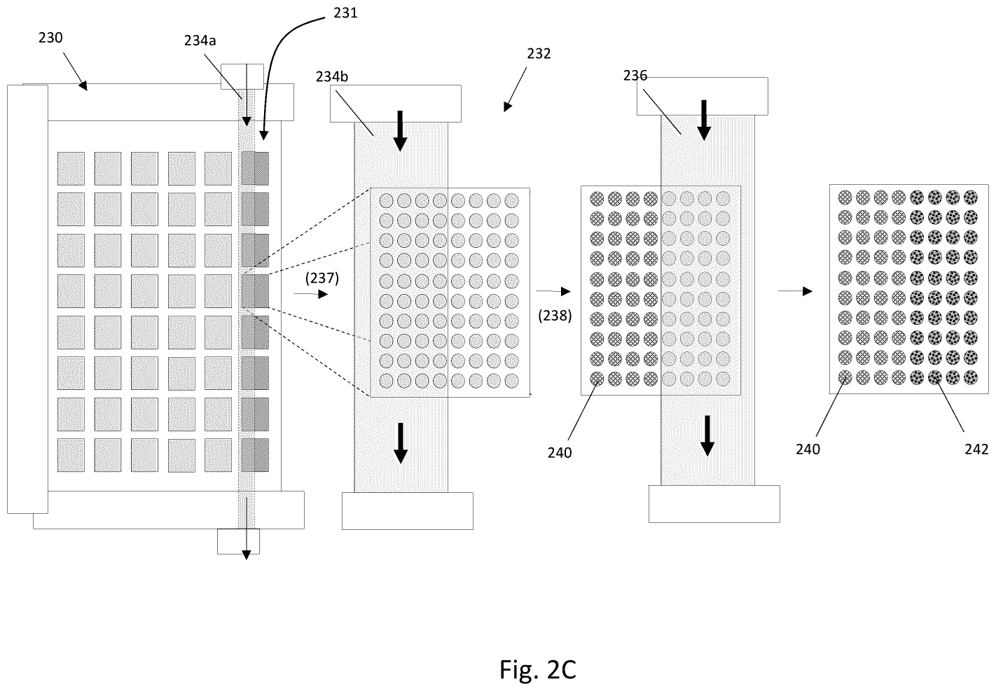

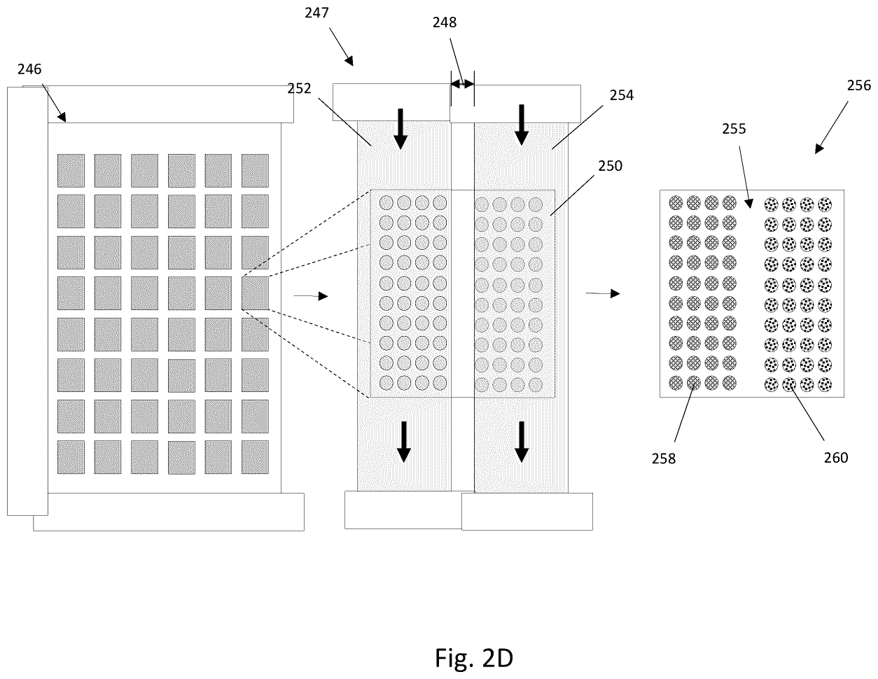

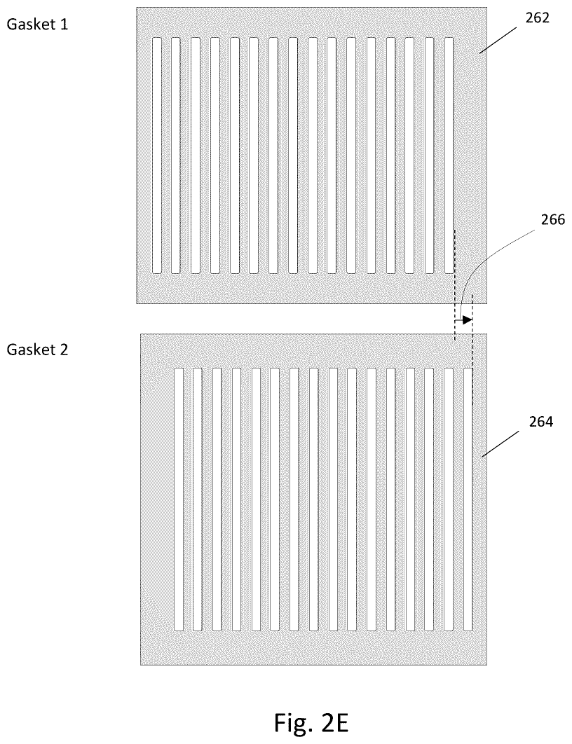

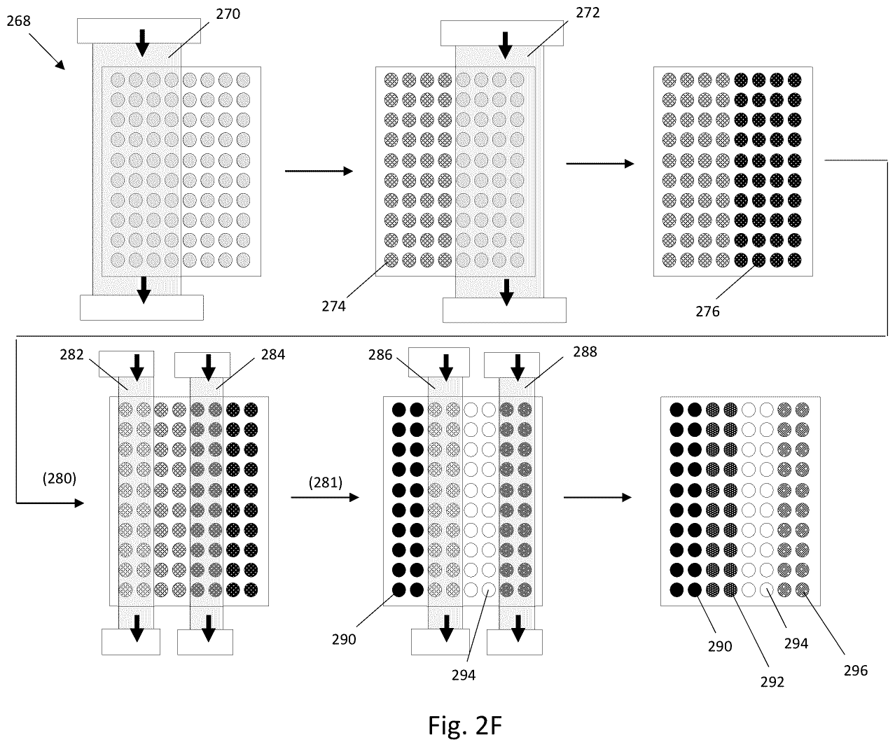

In some embodiments, an array of first oligonucleotide arrays is synthesized (or disposed) on a surface, e.g. as illustrated in FIG. 2A, after which second and third oligonucleotides are attached by forming orthogonal channels for delivering the oligonucleotides (e.g. as illustrated in FIGS. 2A-2B). In some embodiments, surface (202) (see FIG. 2A) may be free of capture oligonucleotides so that the interstitial space (203) between arrays (and between spots or reaction sites within arrays) are free of barcodes. In other embodiments, surface (202) may be coated with capture oligonucleotides for capturing the various barcode oligonucleotides (first, second or third), which may be followed by either extension or ligation to form a combinatorial barcode. In other words, in some embodiments, surface functionalities may comprise capture oligonucleotides. In such latter embodiments, barcoded surfaces may be produced wherein the interstitial spaces (e.g. 203) in an array of arrays contain barcodes of one or more segments. In some embodiments, the ordering of channel delivery and droplet delivery of the first, second and third oligonucleotides may differ. In some embodiments, an array of array of first oligonucleotides is delivered by droplets, followed by channel delivery of second oligonucleotides and third oligonucleotides. In other embodiments, first oligonucleotides are delivered by channel, an array of arrays of second oligonucleotides is delivered by droplets, and third oligonucleotides are delivered by channel. In still other embodiments, first oligonucleotides are delivered by channel, second oligonucleotides are delivered by channel, and an array of arrays of third oligonucleotides is delivered by droplets.

FIG. 1A illustrates one embodiment for sequentially linking three barcode segments to form a spatial barcode for a surface, after which a sample nucleic acid may be captured (i.e. the fourth format described above: BC₁-BC₂-BC₃-sample NA). In one embodiment, first oligonucleotide (102) comprising first barcode segment (BC₁)(106) and sequence (S₁)(104) is attached to surface (100) by its 5′ end by any of a variety of linkages well-known to those skilled in the art, e.g. Beaucage, Curr. Med. Chem., 8(10): 1213-1244 (2001); Frydrych-Tomczak et al, BioTechnologia, 95(1): 5-16 (2014); Ratajczak et al, Methods Mol. Biol., 1368: 25-36 (2016); Uszczynska et al, LabChip, 12(6): 1151-1156 (2012): and the like. Such linkages are formed by reaction of a surface functionality and a complementary functionality of the oligonucleotide being attached. In some embodiments, oligonucleotides being attached, such as, capture oligonucleotides or barcode oligonucleotides, are attached by their 5′ ends, for example, so that their 3′ ends remain free for later extension by a polymerase.

In accordance with some embodiments, first oligonucleotides (102) having different barcode sequences are delivered to separate known locations in an array using a DNA printing device, such as a device manufactured by M2 Automation (Berlin, Germany), Scienion (Berlin, Germany), or the like. In some embodiments, inkjet delivery systems may be used to construct the plurality of arrays, e.g. Cartesian Technologies (Irvine, CA); Barczak et al, Genome Research, 13: 1775-1785 (2003); and the like. In some embodiments, first oligonucleotides of the plurality of arrays may be synthesized in situ using a variety of array synthesis technologies, e.g. Singh-Gasson et al, Nature Biotechnology, 17: 974-978 (1999); Horgan et al, International patent application WO2022/013094; Le, Recent Progress in Ink Jet Technologies II, chapter 1 (1999); Hughes et al, Nature Biotechnology, 19:342-347 (2001); and the like. In some embodiments, such arrays comprise spatially compact rectilinear or hexagonal arrays of non-overlapping, i.e. spatially discrete, reaction sites substantially uniformly coated with first oligonucleotides (102). In some embodiments, arrays of such reaction sites may have, but are not limited to, pitches (center-to-center distances) in the range of from 50-500 μm and diameters in the range of from 30-100 μm. Returning to FIG. 1A, the first oligonucleotides (102) attached to surface (100) can be hybridized (or annealed (108)) to second oligonucleotides (110) comprising segments S₁′ (complementary to segment S₁ (104)), second barcode segment BC₂, and segment S₂′. Afterwards, reagents may be introduced to extend first oligonucleotide (102) so that BC₂ and S₂′ of second oligonucleotide (110) are copied to form a first conjugate surface oligonucleotide. In alternative embodiments, second barcode segment (113) may be attached to first oligonucleotide (102) by ligating a second oligonucleotide using, for example, a ligase, to thereby form a first conjugate surface oligonucleotide. In some alternative embodiments, successive oligonucleotide segments may be attached by ligation using a ligase and splint oligonucleotides that form a duplex with the two oligonucleotides to be ligated. In further embodiments, successive oligonucleotide segments may be attached by ligation using a circligase.

A method of delivering second oligonucleotides (110) and reagents for extending first oligonucleotides (102) is illustrated in FIGS. 2A-2B. After hybridization and extension (112), hybridized and copied second oligonucleotide (110) can be melted (114) from strand (113). Strand (113) (sometimes referred to herein as the “first conjugate surface oligonucleotide”) can be annealed (116) to third oligonucleotide (118) comprising segment S₂′ (complementary to segment S₂ of strand (113)), third barcode segment (BC₃) and segment S₃′. After extension (120) and washing and melting (122), the result is spatial barcode (124) comprising barcode segments BC₁, BC₂ and BC₃, the combination of which may be unique for each reaction site in the plurality of arrays. In some embodiments, segment S₃ (125) may serve as a capture oligonucleotide. For example, it may be a polyT sequence for capturing poly A-tailed messenger RNAs from cells of a sample being analyzed on surface (100). Similar to above, in alternative embodiments, third barcode segment (118) may be attached to strand (113) by ligation.

FIG. 1B illustrates an alternative embodiment that employs tagmentation to attach a third barcode component (thereby forming a barcoded sample nucleic acid of the third format above, namely: BC₁-BC₂-sample NA-BC₃. A review of the tagmentation technique is given in Adey, Genome Research, 31: 1693-1705 (2021); U.S. Pat. Nos. 9,115,396; 9,085,801; 11,319,534; and the like, which are incorporated herein by reference. Barcode segments BC₁-S₁ and BC₂-S₂ (150) can be assembled as described in FIG. 1A where segment S₂ is a capture probe (for example, a polyT segment specific for poly A messenger RNA of a biological sample). Biological sample (151) can be contacted with surface (100) so that polyA mRNA contained therein anneals (152) to capture oligonucleotides (S₂, 153), wherein the mRNA comprises poly A segment (154) and coding segment (156). After extension with a reverse transcriptase and optional template switching, double stranded structure (159) may be obtained, after which it is subjected to tagmentation (160) to attach final barcode segment, BC₃, to give final sequence (162). Using similar procedures, each of the formats (-sample NA-BC₁-BC₂-BC₃; -BC₁-sample NA-BC₂-BC₃; -BC₁-BC₂-sample NA-BC₃; or -BC₁-BC₂-BC₃-sample NA) may be synthesized.

In accordance with some embodiments described herein, second and third oligonucleotides comprising second and third barcode segments, respectively, are delivered to the plurality of arrays by channels as illustrated in FIGS. 2A-2B. Alternative embodiments for delivering and conjugating to first oligonucleotides (or the conjugates of first and second oligonucleotides) may comprise the use of photo-masks and photo-activated ligation, for example, as taught by van Dam, Thesis (California Institute of Technology, 2005). As shown in FIG. 2A, a plurality of arrays (e.g. 204) can be synthesized on surface (202) of slide, or substrate, (200). In this illustration, the plurality of arrays is 240, arranged in a 24×10 rectilinear format. The spacing of the arrays on surface (202) is exaggerated for the sake of illustration. Blow-up (206) of an array shows a 32×24 array of reaction sites (208). In some embodiments, each array of the plurality has the same first oligonucleotides in the same positions. Thus, for example, the sequence of the barcode segment of first oligonucleotide at row 18 and column 11 of array (205) is the same as that of the first oligonucleotide at row 18 and column 11 of array (204). That is, in some embodiments, each of the arrays of a plurality comprise the same first oligonucleotides.

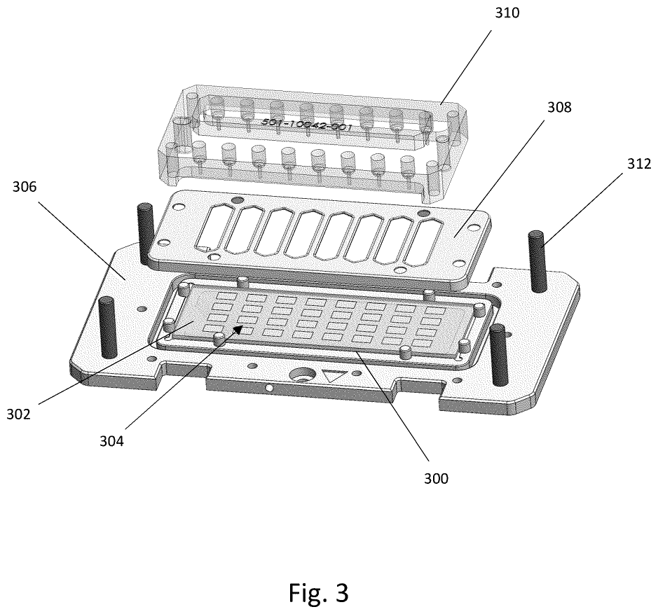

In this embodiment, second oligonucleotides and associated extension reagents (for example, DNA polymerases, reaction buffers, dNTPs, and so on) are delivered by way of channels formed in a layer of material (for example, an elastomeric plastic, or the like), forming a channel body or template that can be placed over the plurality of arrays and partitions it into a plurality of rows or a plurality of columns. One of ordinary skill in the art would understand that the pluralities of arrays, rows, columns, first channels, second channels, and the like, are independent quantities; that is, the values of the pluralities for these separate features need not be the same in any particular embodiment. As illustrated in FIG. 2A, channel template (210) can be placed (212) on surface (202) to partition the plurality of arrays into a plurality of 24 rows of 10 arrays each. Placement of channel template (210) on surface (202) may be implemented using a simple appliance similar to that illustrated in FIG. 3, which sandwiches channel template (210) between surface (202) of substrate (203) and cover (207). Channel templates may vary widely in design and composition depending on the magnitude and arrangement of a plurality of arrays, the size and arrangement of arrays of reaction sites, and the methods used to couple first, second and third oligonucleotides. Channel templates may be fabricated from wide variety of materials well-known in the microfluidics field, such as, silicon, glass, plastic, or the like, e.g. Ren et al, Acc. Chem. Res., 46(11): 2396-2406 (2013). In some embodiments, channel templates may comprise a plastic, such as, polystyrene, polyethylenetetraphthalate glycol, polyethylene terephthalate, polymethylmethacrylate, polyvinylchloride, polycarbonate, thermo plastic elastomer or the like. Guidance in the selection of plastics and fabrication methodologies may be found in the following references: Becker et al, Talanta, 56: 267-287 (2002); Fiorini et al, Biotechniques, 38(3): 429-446 (2005); Bjornson et al, U.S. Pat. No. 6,803,019; Soane et al, U.S. Pat. No. 6,176,962; Schaevitz et al, U.S. Pat. No. 6,908,594; Neyer et al, U.S. Pat. No. 6,838,156; and the like, which references are incorporated herein by reference.

As illustrated in cross-sectional view (216), along median (214) of channel (213), after assembly of substrate (203), channel template (210) and cover (207) exclusive flow paths (211) are created for each row of arrays. Thus, each array of a given row may receive the same second oligonucleotide. In some embodiments, the sequence of the barcode segment of each second oligonucleotide of a different row is different, so that the sequence of second barcode segments uniquely identifies the row on which a spatial barcode is located.

After second oligonucleotides are delivered and coupled to first oligonucleotides, row channel template (210) can be removed. As illustrated in FIG. 2B, column channel template (220) can be placed (224) on surface (202) of substrate (203) to partition the plurality of arrays into a plurality of 10 columns (e.g. 222) of 24 arrays each. As with the partition into rows, channel template (220) may create an exclusive flow path for each column, which permits the arrays of each column to be exposed to the same third oligonucleotide. In some embodiments, the sequence of the barcode segment of each third oligonucleotide of a different column is different, so that the sequence of third barcode segments uniquely identifies the column on which a spatial barcode is located. After coupling of the third oligonucleotide (226), a spatially barcoded surface can be created with spatial barcodes of the form shown in blow-up (228).

In some embodiments, the number of unique barcodes on a surface may be increased by providing channels that are coincident with subsets of reaction sites of the rows or columns of arrays. FIG. 2C illustrates an example of this embodiment for the columns of array (232) shown in a blow-up view with respect to array of arrays (230). In this embodiment, widths of channels (e.g. 234a and 234b in blow-up) are fabricated so that the channels are coincident with half of the spots (or reaction sites) of the arrays of column (231, darker shaded sub-arrays), so that barcode oligonucleotides may be attached (237) sequentially to a first half of reaction sites (e.g. 240) and then (238) to a second half of reaction sites (e.g. 242). This may be accomplished by using two different channel templates with channel positions off-set by (for example) half an array width, or by moving a single channel template a half array width. FIG. 2E illustrates the case wherein two channel templates (262 and 264) (or the gasket components of such channel templates) are used with channels off-set by predetermined amount (266) so that different reaction sites are exposed to reagents delivered by the channels. After such steps, array of arrays (230) will have twice the number of different spatial barcodes than embodiments in which each entire sub-array (e.g. 232) is coincident with the channels delivering the barcode oligonucleotides. In some embodiments, such as shown in FIG. 2D, channel templates may be fabricated so that the halves of the sub-arrays are coincident with different channels, wherein space (248) is selected so that a wall of the channel template can be fitted without covering or obstructing reaction sites. This configuration increases the speed of fabrication and simplifies the oligonucleotide deposition process, so that, for example, different barcode segments are simultaneously added to each half of reaction sites giving products (258 and 260) in a single sub-array (256). This embodiment is facilitated by forming sub-arrays with gap (255) in sub-array (256) which separates the halves and provides space for the wall of the channel template. In other embodiments, sub-arrays may be formed with multiple gaps, for example, dividing reaction sites into three regions instead of two, and such gaps may be formed in both horizontal and vertical directions, for example, for the attachment of second and third oligonucleotides, respectively.

In some embodiments, as illustrated in FIG. 2F, spatial barcode oligonucleotides may be formed combinatorially by delivering their components with channels of different widths. For example, channel (270) may be established by attaching a first channel template to a surface comprising sub-array (268), which delivers barcode oligonucleotides which react with either the surface or a conjugate surface oligonucleotide to give product (e.g. 274) in half of the reaction sites of sub-array (268). Subsequently, channel (272) may be established by attaching a second channel template which delivers barcode oligonucleotides which react with either the surface or a conjugate surface oligonucleotide to give product (e.g. 276) in the other half of the reaction sites of sub-array (268). Next, channels (282 and 284) are established by attaching a third channel template with narrower spaced apart channels that deliver (280) reagents to a first and third quadrants of reaction sites of sub-array (268), which after reacting give products (290) and (294). After step (280), channels (286 and 288) are established by attaching a fourth channel template with spaced apart channels that deliver (281) reagent to a second and fourth quadrants of reaction sites of sub-array (268), which after reacting give products (292 and 296). In such embodiments, barcoded surfaces with four times the number of unique barcodes may be produced as compared to embodiments wherein channels are coincident with entire sub-arrays.

As mentioned above, channel templates (210) and (220) may be applied to surface (202) using an appliance as illustrated in FIG. 3, or like apparatus. Substrate (300) comprising surface (302) with plurality of arrays (304) can be placed in base (306) and channel template (308) can be placed on top creating a partition of rows (or columns) of arrays. On top of channel template (308), manifold (310) may be placed for providing conduits from reagent reservoirs or plates to the channels created by channel template (308). Finally, a top plate (not shown) may be aligned by alignment pins (312) and places on top to complete the assembly.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6th edition (Saunders, 2007).

“Barcode” means a molecular label or identifier. In some embodiments, a barcode is a molecule attached to an analyte or a segment of an analyte (for example, in the case of polynucleotide barcodes and analytes) which may be used to identify the analyte. In some embodiments, a barcode (referred to herein as a “spatial barcode”) may be attached to a surface to identify a location on the surface. In some embodiments, populations of identical spatial barcodes may be disposed within a particular area on a surface. The size and shape of such areas may vary widely. In some embodiments, areas with unique spatial barcodes have the same magnitude and are disposed in a regular pattern on a surface with a density of spatial barcodes per unit area. In some embodiments, densities of such barcodes may vary form 1 barcode per mm² to 1000 barcodes per mm², or from 1 barcode per mm² to 500 barcodes per mm², or from 1barcode to 200 barcodes per mm². In some embodiments, there may be a one-to-one correspondence between different spatial barcodes and different areas on a surface; that is, each different area may have a different and unique barcode. In some embodiments, the identity of a spatial barcode is determinable, for example, by sequencing whenever a spatial barcode is a polynucleotide. In some embodiments, a spatial barcode is an oligonucleotide. In some embodiments, an oligonucleotide spatial barcode comprises a random sequence oligonucleotide. A random sequence oligonucleotide is typically synthesized by a “split and mix” synthesis techniques, for example, as described in the following references that are incorporated herein by reference: Church, U.S. Pat. No. 4,942,124; Godron et al, International patent publication WO2020/120442; Seelig et al, U.S. patent publication 2016/0138086; and the like. Sometimes a random oligonucleotide is represented as “NNN . . . N.” In some embodiments, the term “barcode” includes composite barcodes; that is, an oligonucleotide segment that comprises sub-segments that identify different objects. For example, a first segment of a composite barcode may identify a particular area of a surface and a second segment of a composite barcode may identify a particular molecule (a so-called “unique molecular identifier” or UMI).

“Microfluidics” device or “nanofluidics” device, used interchangeably herein, each means an integrated system for capturing, moving, mixing, dispensing or analyzing small volumes of fluid, including samples (which, in turn, may contain or comprise cellular or molecular analytes of interest), reagents, dilutants, buffers, or the like. Generally, reference to “microfluidics” and “nanofluidics” denotes different scales in the size of devices and volumes of fluids handled. In some embodiments, features of a microfluidic device have cross-sectional dimensions of less than a few hundred square micrometers and have passages, or channels, with capillary dimensions, e.g. having cross-sectional dimensions of from about 1-2 mm to about 0.1 μm. In some embodiments, microfluidics devices have volume capacities in the range of from 100 μL to a few nL, e.g. 10-100 nL or in the range of from 100 μL to 1 μL. Dimensions of corresponding features, or structures, in nanofluidics devices are typically from 1 to 3 orders of magnitude less than those for microfluidics devices. One skilled in the art would know from the circumstances of a particular application which dimensionality would be pertinent. In some embodiments, microfluidic or nanofluidic devices have one or more chambers, ports, and channels that are interconnected and in fluid communication and that are designed for carrying out one or more reactions or processes, either alone or in cooperation with an appliance or instrument that provides support functions, such as sample introduction, fluid and/or reagent driving means, such as positive or negative pressure, acoustical energy, or the like, temperature control, detection systems, data collection and/or integration systems, and the like. In some embodiments, microfluidics and nanofluidics devices may further include valves, pumps, filters and specialized functional coatings on interior walls, e.g. to prevent adsorption of sample components or reactants, facilitate reagent movement by electroosmosis, or the like. Such devices may be fabricated as an integrated device in a solid substrate, which may be glass, plastic, or other solid polymeric materials, and may have a planar format for ease of detecting and monitoring sample and reagent movement, especially via optical or electrochemical methods. In some embodiments, such devices are disposable after a single use. In some embodiments, microfluidic and nanofluidic devices include devices that form and control the movement, mixing, dispensing and analysis of droplets, such as, aqueous droplets immersed in an immiscible fluid, such as a light oil. The fabrication and operation of microfluidics and nanofluidics devices are well-known in the art as exemplified by the following references that are incorporated by reference: Ramsey, U.S. Pat. Nos. 6,001,229; 5,858,195; 6,010,607; and 6,033,546; Soane et al, U.S. Pat. Nos. 5,126,022 and 6,054,034; Nelson et al, U.S. Pat. No. 6,613,525; Maher et al, U.S. Pat. No. 6,399,952; Ricco et al, International patent publication WO 02/24322; Bjornson et al, International patent publication WO 99/19717; Wilding et al, U.S. Pat. Nos. 5,587,128; 5,498,392; Sia et al, Electrophoresis, 24: 3563-3576 (2003); Unger et al, Science, 288: 113-116 (2000); Enzelberger et al, U.S. Pat. No. 6,960,437; Cao, “Nanostructures & Nanomaterials: Synthesis, Properties & Applications,” (Imperial College Press, London, 2004); Haeberle et al, LabChip, 7: 1094-1110 (2007); Ren et al, Acc. Chem. Res., 46(11): 2396-2406(2013); Cheng et al, Biochip Technology (CRC Press, 2001); and the like.

1.-64. (canceled)

65. A method of making a spatially barcoded surface, comprising:
(a) partitioning a surface of a solid support into rows by sealingly attaching to the surface a first channel template comprising a plurality of first channels, wherein the surface comprises a plurality of arrays of first oligonucleotides,
wherein each array of the plurality of arrays comprises a plurality of discrete reaction sites, wherein each discrete reaction site comprises a first oligonucleotide of the first oligonucleotides attached to the surface by a 5′-end of the first oligonucleotide,
wherein the plurality of arrays are arranged in orthogonal rows and columns, and
wherein within each array of the plurality of arrays, each of the first oligonucleotides comprises a first barcode segment comprising a first barcode sequence associated with a location of the discrete reaction site within the array, and wherein each discrete reaction site of an array comprises a unique first barcode sequence within the array; and
(b) reacting second oligonucleotides loaded into each first channel of the first channel template with the first oligonucleotides to form first spatial barcode oligonucleotides, wherein each first channel of the first channel template comprises a second oligonucleotide of the second oligonucleotides that comprises a second barcode segment, wherein each first channel of the first channel template comprises a unique second barcode sequence,
wherein the rows of the plurality of arrays are at least partially coincident with the first channels of the first channel template.

66. The method of claim 65, wherein each first spatial barcode oligonucleotide uniquely identifies the spatial location of its discrete reaction site for a plurality of arrays within a column.

67. The method of claim 65, further comprising:
(c) partitioning the surface into columns by sealingly attaching to the surface a second channel template comprising a plurality of channels; and
(d) reacting third oligonucleotides loaded into each channel of the second channel template with the first spatial barcode oligonucleotides to form second spatial barcode oligonucleotides, wherein each channel of the second channel template comprises a third oligonucleotide of the third oligonucleotides that comprises a third barcode segment, wherein each channel of the second channel template comprises a unique third barcode sequence,
wherein the columns of the plurality of arrays are at least partially coincident with the columns of the second channel template.

68. The method of claim 65, wherein prior to (a), each array of the plurality of arrays includes a same first oligonucleotide sequence of the first oligonucleotide in a same position of the discrete reaction site within the array.

69. The method of claim 65, wherein each array of the plurality of arrays comprises a pitch between the discrete reaction sites from about 50 μm to about 500 μm.

70. The method of claim 65, wherein the discrete reaction sites each comprise a diameter from about 30 μm to about 300 μm.

71. The method of claim 65, wherein each array of the plurality of arrays comprises from about 50 to about 200 discrete reaction sites per mm².

72. The method of claim 65, wherein the reacting in (b) comprises extending the first oligonucleotide over the second oligonucleotide with a DNA polymerase.

73. The method of claim 65, wherein the reacting in (b) comprises ligating the first oligonucleotides to the second oligonucleotides.

74. The method of claim 73, wherein the ligating comprises hybridizing the first and second oligonucleotides to splint oligonucleotides, thereby forming duplexes comprising the first oligonucleotides and the second oligonucleotides.

75. The method of claim 65, wherein the second oligonucleotides comprise capture probes configured to hybridize to target nucleic acids.

76. The method of claim 75, wherein the capture probes comprise polyT sequences.

77. The method of claim 75, wherein the first spatial barcode oligonucleotides comprise, from 5′ to 3′, the first barcode sequence, the second barcode sequence, and the capture probe.

78. The method of claim 65, further comprising removing the first channel template from the surface.

79. The method of claim 65, further comprising synthesizing the plurality of arrays, wherein the synthesizing comprises depositing the first oligonucleotides on the surface to form the discrete reaction sites.

80. The method of claim 65, further comprising synthesizing the plurality of arrays, wherein the synthesizing comprises reacting surface functionalities with complementary functionalities on the first oligonucleotides to form covalent bonds between the first oligonucleotides and the surface.

81. The method of claim 65, wherein the surface is planar.

82. The method of claim 65, wherein the surface comprises glass, plastic, silicon, metal oxides, or a combination thereof.

83. The method of claim 65, wherein each of the first spatial barcode oligonucleotides further comprises a unique molecular identifier sequence.

84. The method of claim 83, wherein the second oligonucleotides comprise the unique molecular identifier sequence or a complement of the unique molecular identifier sequence.

85. A flow cell comprising a channel and a surface, wherein the surface of the channel comprises a plurality of arrays of spatial barcode oligonucleotides,
wherein each array of the plurality of arrays comprises a plurality of discrete reaction sites, wherein each discrete reaction site comprises a spatial barcode oligonucleotide of the plurality of spatial barcode oligonucleotides attached to the surface by a 5′-end,
wherein the plurality of arrays are arranged in orthogonal rows and columns,
wherein within each array of the plurality of arrays, each of the spatial barcode oligonucleotides of the plurality of spatial barcode oligonucleotides within each array comprises a barcode segment comprising a first barcode sequence associated with a location of the discrete reaction site, wherein each discrete reaction site of an array comprises a unique first barcode sequence within the array, and
wherein each array of the plurality of arrays in the channel comprises a spatial barcode oligonucleotide of the plurality of spatial barcode oligonucleotides that comprises a second barcode sequence associated with a location of an array within the channel.

86. The flow cell of claim 85, wherein each spatial barcode oligonucleotide of the plurality of spatial barcode oligonucleotides further comprises a capture probe configured to hybridize to a target nucleic acid.

87. The flow cell of claim 86, wherein the capture probe comprises a polyT sequence.

88. The flow cell of claim 86, wherein each spatial barcode oligonucleotide of the plurality of spatial barcode oligonucleotides comprises, from 5′ to 3′, the first barcode sequence, the second barcode sequence, and the capture probe.

89. The flow cell of claim 85, wherein each spatial barcode oligonucleotide of the plurality of spatial barcode oligonucleotides further comprises a unique molecular identifier sequence.
